(19) United States
(12) Patent Application Publication
(10) Pub. No.: US 2012/0315259 A1
(43) Pub. Date: Dec. 13, 2012

(54) METHOD AND COMPOSITION FOR SKIN CARE COMPRISING CORD BLOOD SERUM OR PLASMA OR COMPONENTS THEREOF

(76) Inventor: Hyman Friedlander, Kiryat Ono (IL)

(21) Appl. No.: 13/579,284

(22) PCT Filed: Feb. 1, 2011
(86) PCT No.: PCT/IB11/50435

§ 371 Date: Aug. 16, 2012

Related U.S. Application Data
(60) Provisional application No. 61/305,559, filed on Feb. 18, 2010.

(51) Int. Cl. A61K 35/12; A61K 38/22; A61K 38/18; A61K 35/28; A61P 17/14; A61K 35/16; A61K 35/50; A61K 35/44; A61P 17/00; A61K 38/43; A61K 35/14

(52) U.S. Cl. 424/93.71; 424/520; 424/94.1; 514/9.7; 514/7.6; 424/577; 424/529; 424/531; 424/530; 424/583; 424/582

(57) ABSTRACT

A method and composition for treating dermatological conditions and improving skin condition and helping hair to grow or thicken involves removing extracts including beneficial secretions from mesenchymal stem cells or other cells with regenerative properties to use itself or components thereof alone or with other skin or hair care reagents as a topical ointment or formula to apply to the skin or hair topically for therapeutic and cosmetic purposes.

This patent application claims the benefit of U.S. Provisional Patent Application No. 61/305,559, filed Feb. 18, 2010

### FIELD AND BACKGROUND OF THE INVENTION

The present invention relates to compositions for skin and hair care products and, more particularly, to skin and hair care compositions that include cord blood serum or plasma or components thereof.

Various dermatological conditions, including but not limited to dermatitis, acne, skin irritation, skin rash and dry skin, affect human skin and there is an obvious need to treat them effectively. In addition, there is a widespread desire for people to retard the effects on their skin from normal aging including by improving skin texture and reducing wrinkles. Protection of the skin from overexposure to ultra violet light and injury to skin from surgical suturing are additional examples in which a need to improve care for the skin is recognized.

With respect to hair, premature or natural hair loss and thinning hair are well known problems that affect both men and women. Various attempts have been made to compose formulae and methods to care for the longevity and quality of the skin, which is an important organ of the body, and to care for the quality and quantity of one's hair, including by enhancing the growth of hair. Such ointments and products have been available for a long time. In addition, numerous products including ointments, creams, gels, powders and other products purport to condition or improve various properties of the skin or hair including their appearance, shine, texture, strength, condition, thickness, smoothness, color, etc.

It would be highly advantageous to have a product for skin care and a product for hair care that has inherent qualities and ingredients that provide active and long lasting care for the skin suffering from skin conditions or improving the youth or other qualities of the skin; and it would be highly advantageous to have a product that improves the qualities of human hair as well as retards or reverses hair loss or thickens thin or thinning hair. It would also be particularly advantageous to have a product or method that would be useful for both skin care and hair care.

### SUMMARY OF THE INVENTION

The current invention provides an improved method and composition for the use of regenerative bio-material in the manufacture of cosmetic as well as pharmacological compositions for skin and hair care. The present invention suggests at least utilizing the umbilical serum and/or plasma, excluding the cellular components, for skin care ointments, pharmaceutical preparations and the like.

Umbilical cord blood may be a better source than adult blood, for therapeutic and cosmetic proteins and reagents, since it is richer in stem cells and also in cytokines and growth factors which support cells and tissue development.

The present invention provides a method for skin care, both therapeutic and cosmetic, including administering of treatment preparations that include different proportions of serum or plasma, or serum components derived from umbilical cord blood, to a subject in need thereof at an effective amount for dermatological conditions, including improving skin texture, reducing winkles, UV protection, anti-aging effect, dry skin, hair support and growth stimulation, surgical sutures, dermatitis, skin rash wound healing etc.

According to the present invention there is provided method, including the steps of: removing from a natural medium an extract, the extract including components that had been secreted into the natural medium from regenerative cells; and applying the extract, or components of the extract, topically to a portion of a skin of a human, to foster growth of skin cells.

According to further features in preferred embodiments of the invention described below the method includes the step of adding the extract, or components of the extract, to a topical skin composition, before applying topically to a portion of skin.

According to still further features in the described preferred embodiments removing the extract involves removing a cell-free extract from the natural medium.

According to another embodiment there is provided a method of improving a quality of human hair that is growing on a human scalp, comprising the steps of: removing from a natural medium, cell-free extracts that had been secreted into the natural medium from regenerative cells; and applying topically the cell-free extracts or components of the cell-free extracts, to the human hair.

According to further features the topical application includes smearing onto the human hair.

According to still further features the topical application involves including the cell-free extracts or components thereof, in a shampoo and shampooing the hair with the shampoo.

method of assisting in growing human hair on a human scalp, including: applying topically to the human scalp a cell-free extract, or components of the cell-free extract; wherein the extract includes components secreted from mesenchymal stem cells, and wherein the extract is removed from a natural medium into which the mesenchymal stem cells had been secreted.

According to another embodiment there is provided a composition for treating dermatological conditions and/or hair conditions, comprising a cell-free extract including components secreted from regenerative cells, and wherein the cell-free extract is removed from a natural medium into which the regenerative cells had secreted the components.

According to further features the regenerative cells are selected from the group consisting of mesenchymal stem cells, endothelial progenitor cells, hematopoietic stem cells, progenitor cells, monocytes, macrophages, keratinocytes and fibroblasts.

According to still further features the secreted components are selected from the group consisting of enzymes, hormones, growth factor, regulatory factors and lymphocytes.

According to still further features the natural medium is selected from the group consisting of bone marrow, fat blood, umbilical cord blood, Wharton's Jelly, placenta, amniotic fluid, cord blood stem cells, cord serum, plasma and whole umbilical cord.

### DESCRIPTION OF THE PREFERRED EMBODIMENTS

The principles and operation of a method and composition for skin and hair care according to the present invention may be better understood with the following description.

### Regenerative Cells

As a matter of definition, the term “regenerative cells” is intended to mean any combination in any proportion of the following: mesenchymal stem cells, endothelial progenitor cells, hematopoietic stem and progenitor cells, monocytes, macrophages, keratinocytes, fibroblasts, and any other cell type, excluding embryonic stem cells, that produces or secretes growth factors or cytokines or regulatory factors. The regenerative cells may be used immediately when donated or may be used only after being frozen for a time.

### Natural Medium

The mesenchymal stem cells and endothelial progenitor cells or their mixtures may be obtained from natural mediums found in the body, such as different tissues in the adult body such as bone marrow, fat blood, umbilical cord blood, or from Wharton's Jelly or placenta or amniotic fluid, from normal deliveries, in which other cord components such as cord blood stem cells or cord serum or plasma have been removed and used for other applications, or from frozen cord blood units stored in public or family cord blood banks. Stem cells and progenitor cells may also be obtained from bone marrow or peripheral blood or mobilized peripheral blood, or cord blood or menstrual blood or fat tissue, or any tissue in the body that may be an efficient source for stem, progenitor or regenerative cells. In some embodiments, such as where the regenerative cells are obtained from an entire umbilical cord without previous removal of blood vessels or any other tissue, mesenchymal stem cells may be mixed together with other types of stem and progenitor cells such as endothelial progenitor cells and fibroblasts. The general term “natural medium” is used hereinafter to describe the aforementioned naturally occurring medium from which the regenerative cells are obtained. It should be noted that the natural medium may be obtained from a single donor, or pooled from multiple donors.

### Beneficial Factors/Secreted Components

The “beneficial factors” or “secreted components” are elements or components that may have been secreted from the regenerative cells into a natural medium. For example, regenerative cells such as mesenchymal stem cells, secrete beneficial factors into a natural medium such as plasma. The beneficial factors/secreted components are intended to refer to factors that have a beneficial effect on other cells of the human body. The following is a not-necessarily exhaustive list of examples of “beneficial factors”/“secreted components”: growth factors, regulatory factors, hormones, enzymes, lymphokines and peptides.

### Method of Extraction and Application of Skin Care

In accordance with one embodiment, the present invention is a method. This method may include the removal of an extract from a natural medium. The extract includes “beneficial factors” which are components that have been secreted from regenerative cells into the natural medium. The extract does not include whole cells [and basic cellular components].

The method may further include a step of applying the extract, and/or isolating and applying components of this extract, topically onto a portion of the skin or in the case of therapeutic use, onto an affected area of the skin, of a human for either therapeutic or cosmetic purposes, such as to foster growth of skin cells or hair cells.

The method may also include a step of adding known cosmetic or therapeutic skin care formulations to the extract, for example to make it more effective, more stable or for other reasons, and only afterwards applying the total formula to the skin. Topical skin compositions may include hand and body creams, hair shampoos, under eye creams, bath gels and soaps, shaving and after shaving lotions and others.

### Method for Extraction and Application of Hair Care

The present invention also embodies a method of improving the quality of human hair that is growing on a human scalp. This embodiment of the method may include a step of extracting cell-free “beneficial factors” (that had been secreted from regenerative cells) from the natural medium and applying the entire extract, or components of the extract, topically to the human hair. The step of applying topically to the human hair may involve smearing onto the hair either at the scalp or onto the hair at points away from the scalp. In some case, applying topically to the human hair may involve including the cell-free extracts in a shampoo and shampooing the hair with that shampoo.

### Skin/Hair Compositions

The present invention may be a composition for treating dermatological conditions and/or hair conditions, comprising a cell-free extract of secretions from regenerative cells. The extract is removed from a natural medium. The natural medium is the medium containing the regenerative cells and the beneficial secretions which the regenerative cells secrete into the natural medium. In one formulation, the regenerative cells may be selected from the group consisting of mesenchymal stem cells, endothelial progenitor cells, hematopoietic stem cells, progenitor cells, monocytes, macrophages, keratinocytes and fibroblasts. In one embodiment, the secretions include growth factor, regulatory factors, hormones, enzymes, lymphokines and peptides.

The composition of the present invention may also include extracts from natural mediums such as placenta extracts, extracts from Wharton's jelly, or amniotic fluids or components extracted from these tissues, or cord blood plasma, cord blood serum or components derived from them.

### Additional Options

It is contemplated that the elements extracted from the natural medium, can be tested for various contaminating agents or toxic materials, and/or tested for efficacy in various in vitro and in vivo models of skin and tissue regeneration or disease models.

In certain embodiments, an apparatus such as a bandage or other carrier may incorporate the extracts or composition of the present invention. The bandage or other carrier material may be used by being applied to the skin or on a skin injury, or to skin diseases for cosmetic or therapeutic applications.

It is also contemplated that the present invention may be administered to a subject in need of food supplement, as well as in combinations with other food supplements or nutritional components.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method, comprising the steps of:
(a) removing from a natural medium an extract, said extract including components that had been secreted into said natural medium from regenerative cells; and
(b) applying said extract, or components of said extract, topically to a portion of a skin of a human, to foster growth of skin cells.

2. The method of claim 1, further comprising the step of:
(c) adding said extract, or said components of said extract, to a topical skin composition, before applying topically to said portion of said skin.

3. The method of claim 1, wherein removing said extract involves removing a cell-free extract from said natural medium.

4. A method of improving a quality of human hair that is growing on a human scalp, comprising the steps of:
(a) removing from a natural medium, cell-free extracts that had been secreted into said natural medium from regenerative cells; and
(b) applying topically said cell-free extracts or components of said cell-free extracts, to the human hair.

5. The method of claim 4, wherein said topical application includes smearing onto the human hair.

6. The method of claim 5, wherein said topically applying involves including said cell-free extracts or components thereof, in a shampoo and shampooing the human hair with the shampoo.

7. A method of assisting in growing human hair on a human scalp, comprising:
applying topically to the human scalp a cell-free extract, or components of said cell-free extract; wherein said extract includes components secreted from mesenchymal stem cells, and wherein said extract is removed from a natural medium into which said mesenchymal stem cells had been secreted.

8. A composition for treating dermatological conditions and/or hair conditions, comprising a cell-free extract including components secreted from regenerative cells, and wherein said cell-free extract is removed from a natural medium into which said regenerative cells had secreted said components.

9. The composition of claim 8, wherein the regenerative cells are selected from the group consisting of mesenchymal stem cells, endothelial progenitor cells, hematopoietic stem cells, progenitor cells, monocytes, macrophages, keratinocytes and fibroblasts.

10. The composition of claim 8, wherein said secreted components are selected from the group consisting of enzymes, hormones, growth factor, regulatory factors and lymphocytes.

11. The composition of claim 8, wherein said natural medium is selected from the group consisting of bone marrow, fat blood, umbilical cord blood, Wharton's Jelly, placenta, amniotic fluid, cord blood stem cells, cord serum, plasma and whole umbilical cord.
